(19) United States
(12) Patent Application Publication
(10) Pub. No.: US 2010/0104634 A1
(43) Pub. Date: Apr. 29, 2010

(54) PHARMACEUTICAL COMPOSITIONS OF ENTACAPONE

(76) Inventor: Mahesh Rameshwar KALANTRI, Beed (IN)

Correspondence Address: BIO INTELLECTUAL PROPERTY SERVICES (BIO IPS) LLC, 8509 KERNON CT., LORTON, VA, 22079, US

(21) Appl. No.: 12/447,426

(22) PCT Filed: Dec. 20, 2007
(86) PCT No.: PCT/IB2007/004023

§ 371 Date: Jul. 7, 2009

(30) Foreign Application Priority Data
Dec. 27, 2006 (IN) 2136/MUM/2006
Jan. 19, 2007 (IN) 114/MUM/2007
Jan. 19, 2007 (IN) 118/MUM/2007

(51) Int. Cl. A61K 31/165; A61P 25/16; A61K 9/48; A61K 9/20; A61K 9/14; A61K 31/724

(52) U.S. Cl. 424/452; 424/400; 424/465; 424/489; 514/58; 514/619

(57) ABSTRACT

The invention relates to pharmaceutical compositions comprising entacapone or pharmaceutically acceptable salts thereof. The invention also relates to processes for the preparation of such compositions.

### FIELD OF THE INVENTION

The invention relates to pharmaceutical compositions comprising entacapone or pharmaceutically acceptable salts thereof. The invention also relates to processes for the preparation of such compositions.

### BACKGROUND OF THE INVENTION

Entacapone, an inhibitor of catechol-O-methyltransferase (COMT), is a nitro-catechol-structured compound with a molecular weight of 305.3. It is used in the treatment of Parkinson's disease as an adjunct to levodopa/carbidopa therapy. The chemical name of entacapone is (E)-2-cyano-3-(3,4-dihydroxy-5-nitrophenyl)-N,N-diethyl-2-propenamide. Its empirical formula is C₁₄H₁₅N₃O₅, and its structural formula is:

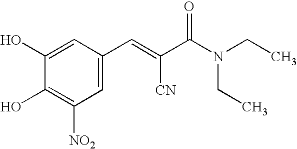

U.S. Pat. No. 4,963,590 discloses a pharmaceutical composition comprising entacapone and pharmaceutically acceptable carrier.

U.S. Pat. No. 6,599,530 provides an oral compacted composition in the form of a tablet, which comprises entacapone, nitecapone, or pharmaceutically acceptable salt of entacapone or nitecapone, and croscarmellose sodium in an amount of at least 6% by weight of the composition.

International (PCT) Publication No. WO2006/131591 discloses oral dosage forms of entacapone and methods of preparation thereof.

Entacapone is classified as a class IV drug according to Biopharmaceutics Classification system (BCS), and poses problems of low solubility, low dissolution rate and consequently low bioavailability. The invention addresses and overcomes these commonly encountered problems.

### SUMMARY OF THE INVENTION

In one general aspect there is provided a method of making a pharmaceutical composition of entacapone. The method includes providing particles of entacapone or salts thereof having a particle size (D₉₀) of 40 microns or less; forming a mixture by mixing the particles of entacapone or salts thereof with one or more pharmaceutically acceptable excipients; and forming the mixture into a pharmaceutical dosage form.

The term “D₉₀ particle size of 40 microns” as used herein refers to particle size distribution wherein at least 90% of particles have size less than 40 microns.

Embodiments of the method of making the pharmaceutical composition may include one or more of the following features. The method may include reducing the particle size of the particles of entacapone in a particle size reducing operation. The particle size reduction may be carried out using one or both of chemical methods and mechanical methods. The particle size reducing operation may reduce the size of the particles of entacapone to have a particle size (D₉₀) that is 40 microns or less.

In another aspect there is provided a pharmaceutical composition that includes particles of entacapone or salts thereof, the particles having a particle size (D₉₀) that is 40 microns or less.

Embodiments of the pharmaceutical composition may include one or more of the following features. For example, the composition has at least 80% dissolution of entacapone or salt thereof within 30 minutes, when it is tested in Apparatus 2 (USP, Dissolution, paddle, 50 rpm) using 900 ml of pH 5.5 phosphate buffer at 37° C.±0.5° C.

The pharmaceutical composition may further include one or more pharmaceutically acceptable excipients selected from the group of fillers, lubricants, disintegrants, surfactants, binders and glidants.

In another general aspect there is provided a pharmaceutical composition that includes granules of entacapone or salts thereof, the granules having a particle size that is 900 microns or less.

In another general aspect there is provided a pharmaceutical composition that includes entacapone or salts thereof and cyclodextrins or derivatives thereof.

Embodiments of the composition may include one or more of the following features. For example, the entacapone or salts thereof may be present in admixture with cyclodextrins or derivatives thereof or present in the form of a complex with cyclodextrins or derivatives thereof.

In another general aspect there is provided a pharmaceutical composition that includes entacapone or salts thereof and a wetting agent.

Embodiments of the pharmaceutical compositions may include one or more of the following features or those described above. For example, the composition may further include one or more pharmaceutically acceptable excipients selected from the group of fillers, lubricants, disintegrants, binders and glidants.

The details of one or more embodiments of the inventions are set forth in the description below. Other features, objects and advantages of the inventions will be apparent from the description and claims.

### DETAILED DESCRIPTION OF THE INVENTION

The inventors have now discovered that when entacapone having a particle size (D₉₀) of 40 microns or less is used, it results in increased solubility, significant increase in percent drug release of entacapone and hence improved bioavailability of entacapone pharmaceutical compositions as compared to entacapone pharmaceutical compositions that contain large sized entacapone particles. The inventors have also discovered that the use of a wetting agent reduces the surface tension of water and therefore increases adhesion of water to the entacapone surface. Improved wettability is observed as a lower contact angle between the entacapone and water, which in turn results in increased solubility, significant increase in percent drug release of entacapone and hence improved bioavailability. It was also discovered that the use of cyclodextrin also results in increased solubility, significant increase in percent drug release of entacapone and consequently improved bioavailability of entacapone.

In general, the entacapone having a particle size (D₉₀) of 40 microns or less may be prepared by chemical methods and/or mechanical methods.

The chemical methods may include one or more of solvent crystallization, chemical synthesis, modified crystal engineering, freeze-drying or other suitable means.

The mechanical methods to reduce the particle size of entacapone may include one or more of milling, ultrasonication or other suitable techniques. The milling may include conventional techniques like ball mill, fluid energy attrition mills, jet mills or other suitable means.

Alternatively, the particle size of entacapone may be reduced by dissolving entacapone of bigger size in a suitable solvent such as dimethylformamide optionally, with other pharmaceutically acceptable excipients and the resultant mass may be spray dried to get the desired particle size of entacapone. The resultant mass may optionally be spray dried over other excipients to form a film.

The particle size of entacapone may also be reduced by co-melting entacapone with other pharmaceutically acceptable excipients and resultant mass may be cooled to get solid-solid dispersion.

The pharmaceutical composition may be prepared by mixing entacapone (D₉₀ particle size of 40 microns or less) with other pharmaceutically acceptable excipients, compacting the pre-mix through a compactor, breaking flakes into granules of the desired size. The compacting and breaking may be carried out one or more times. The granules may be mixed with one or more of a lubricant, disintegrant, glidant, or a mixture thereof, and the mixture may be formulated into a suitable dosage form.

The pharmaceutical composition may also be prepared by mixing entacapone with one or more wetting agents and other pharmaceutically acceptable excipients, compacting the pre-mix through a compactor, breaking flakes into granules of the desired size. The compacting and breaking may be carried out one or more times. The granules may be mixed with one or more of a lubricant, disintegrant, glidant or a mixture thereof, and the mixture may be formulated into a suitable dosage form.

The “wetting agent” may be one or more of anionic, cationic or non-ionic surface-active agents or surfactants. The wetting agent may further include one or more of gum acacia, guar gum, xanthan gum, kaolin, bentonite, hectorite, tragacanth, sodium alginate, pectin, and the like.

Suitable anionic surfactants may include one or more of sodium dodecyl sulfate (SDS), sodium lauryl sulfate (SLS), sodium laurate, dialkyl sodium sulfosuccinates, sodium stearate, potassium stearate, sodium oleate, and the like.

Suitable cationic surfactants may include one or more of benzalkonium chloride, bis-2-hydroxyethyl oleyl amine, benzethonium chloride, cetrimide, and the like.

Suitable non-ionic surfactants may include one or more of polyoxyethylene sorbitan fatty acid esters, fatty alcohols such as lauryl, cetyl and stearyl alcohols; glyceryl esters such as the naturally occurring mono-, di-, and tri-glycerides; fatty acid esters of fatty alcohols and other alcohols such as propylene glycol, polyethylene glycol, sorbitan, cholesterol, and the like.

The pharmaceutical composition may be prepared by mixing entacapone with cyclodextrin and other pharmaceutically acceptable excipients, compacting the pre-mix through a compactor, breaking flakes into granules of the desired size. The compacting and breaking may be carried out one or more times. The granules may be mixed with one or more of a lubricant, disintegrant, glidant or a mixture thereof, and the mixture may be formulated into a suitable dosage form.

The complex of entacapone and cyclodextrin may be prepared by various processes including solvent evaporation, kneading, spray drying, colloidal milling, high speed mixing, trituration or simple mixing. The entacapone may be present in an amount relative to the cyclodextrin, such that a molar ratio between the entacapone and the cyclodextrin may be from about 1:1 to 1:10.

Suitable water soluble cyclodextrin derivatives may include one or more of, β-cyclodextrin, α-cyclodextrin, γ-cyclodextrins, hydroxypropyl-α-cyclodextrin, hydroxypropyl-β-cyclodextrin, dimethyl-β-cyclodextrin, 2-hydroxyethyl β-cyclodextrin, trimethyl-β-cyclodextrin, sulfonated cyclodextrins and the like.

The pharmaceutical composition as described herein may include other pharmaceutically acceptable excipients. Examples of other pharmaceutically acceptable as used herein include binders, fillers, lubricants, disintegrants, glidants, and the like.

Suitable binders may include one or more of povidone, starch, stearic acid, gums, hydroxypropylmethylcellulose, and the like.

Suitable fillers may include one or more of microcrystalline cellulose, lactose, mannitol, calcium phosphate, calcium sulfate, kaolin, dry starch, powdered sugar, and the like.

Suitable lubricants may include one or more of magnesium stearate, zinc stearate, calcium stearate, stearic acid, sodium stearyl fumarate, hydrogenated vegetable oil, and the like.

Suitable glidants may include one or more of colloidal silicon dioxide, talc or cornstarch, and the like.

Suitable disintegrants may include one or more of starch, croscarmellose sodium, crosspovidone, sodium starch glycolate, and the like.

The pharmaceutical compositions of the invention may be formulated into monolayered tablets, bilayered tablets, tablet in a tablet, a caplet, minitablets, capsules, tablet in a capsule, granules in capsules, pellets, pellets in capsules, or powder. Further, the powder or granules may be suspended to give a pharmaceutically acceptable oral suspension.

The pharmaceutical composition as described herein may include granules of entacapone having a size of 900 microns or less.

The pharmaceutical composition that includes the particles of entacapone having a size (D₉₀) that is 40 microns or less may exhibit a dissolution profile such that within 30 minutes, at least 80% of entacapone or salt thereof is released, wherein the release rate is measured in Apparatus 2 (USP, Dissolution, paddle, 50 rpm) using 900 ml of pH 5.5 phosphate buffer at 37° C.±0.5° C.

The invention is further illustrated by the following examples which are provided merely to be exemplary of the invention and do not limit the scope of the invention. Certain modifications and equivalents will be apparent to those skilled in the art and are intended to be included within the scope of the invention.

### Example 1

Table 1 provides the composition of batches

TABLE 1

| S. No. | Ingredients | Qty/tablet (% w/w) |
|---|---|---|
| 1 | Entacapone | 15-50 |
| | (D₉₀ particle size of 40 | |
| | microns or less) | |
| 2 | Microcrystalline cellulose | 20-65 |
| 3 | Mannitol | 5-50 |
| 4 | Croscarmellose sodium | 2-5 |
| 5 | Colloidal silicon dioxide | 0.5-2 |
| 6 | Sodium starch glycollate | 2-12 |
| 7 | Hydrogenated vegetable oil | 0.5-6 |
| 8 | Talc | 0.5-2 |
| 9 | Magnesium stearate | 0.5-2 |
| 10 | Opadry | 0.5-5 |

Procedure: Entacapone (D₉₀ particle size of 40 microns or less), microcrystalline cellulose, mannitol, croscarmellose sodium and colloidal silicon dioxide were sieved and mixed together in a double cone blender. Magnesium stearate was mixed with above pre-mix in a double cone blender. Half of this mixture was compacted through a roll compactor and milling was carried out to break flakes in to granules using a multi mill. The remaining half of the mixture was also compacted through a roll compactor along with fines of first half and again milling was done using a multimill to obtain granules of desired size. The granules were mixed with hydrogenated vegetable oil, sodium starch glycollate, colloidal silicon dioxide and talc. The granules were then lubricated with magnesium stearate and the final blend was compressed in to tablets using suitable tooling and coated with aqueous dispersion of Opadry.

Table 2 provides the comparative dissolution data for entacapone tablets (D₉₀ particle size of 40 microns vis-à-vis D₉₀ particle size of 40 microns or less) prepared as per the formula given in Table 1. For determination of drug release rate, USP Type 2 Apparatus (rpm 50) was used wherein 900 ml of pH 5.5 phosphate buffer at 37° C.±0.5° C. was used as a medium.

TABLE 2

| | Entacapone tablets | Entacapone tablets |
|---|---|---|
| | (Entacapone with D₉₀ particle | (Entacapone with D₉₀ particle |
| Time | size of 40 microns) | size of 40 microns) |
| (min) | % drug released | % drug released |
| 20 | 61 | 68 |
| 30 | 66 | 83 |
| 45 | 72 | 89 |

### Example 2

Table 3 provides the composition of batches.

TABLE 3

| S. No. | Ingredients | Qty/tablet (% w/w) |
|---|---|---|
| 1 | Entacapone | 15-50 |
| 2 | Microcrystalline cellulose | 20-65 |
| 3 | Mannitol | 5-50 |
| 4 | Sodium dodecyl sulfate | 0.5-6 |
| 4 | Croscarmellose sodium | 2-5 |
| 5 | Colloidal silicon dioxide | 0.5-2 |
| 6 | Sodium starch glycollate | 2-12 |
| 7 | Hydrogenated vegetable oil | 0.5-6 |
| 8 | Talc | 0.5-2 |
| 9 | Magnesium stearate | 0.5-2 |
| 10 | Opadry | 0.5-5 |

Procedure: Entacapone, mannitol and sodium dodecyl sulfate were co-sifted and mixed with microcrystalline cellulose, croscarmellose sodium and colloidal silicon dioxide in double cone blender. Magnesium stearate was mixed with above pre-mix in a double cone blender. Half of this mixture was compacted through a roll compactor and sizing was carried out to break flakes in to granules using a multi mill. The remaining half of the mixture was also compacted through a roll compactor along with fines of first half and sizing was done using a multimill to obtain granules of desired size. The granules were mixed with hydrogenated vegetable oil, sodium starch glycollate, colloidal silicon dioxide and talc. The granules were then lubricated with magnesium stearate and the final blend was compressed in to tablets using suitable tooling and coated with aqueous dispersion of Opadry.

### Example 3

Table 4 provides composition of batches.

TABLE 4

| S. No. | Ingredients | Qty/tablet (% w/w) |
|---|---|---|
| 1 | Entacapone | 15-50 |
| 2 | Microcrystalline cellulose | 20-65 |
| 3 | Mannitol | 5-50 |
| 4 | Beta cyclodextrin | 20-65 |
| 4 | Croscarmellose sodium | 2-5 |
| 5 | Colloidal silicon dioxide | 0.5-2 |
| 6 | Sodium starch glycollate | 2-12 |
| 7 | Hydrogenated vegetable oil | 0.5-6 |
| 8 | Talc | 0.5-2 |
| 9 | Magnesium stearate | 0.5-2 |
| 10 | Opadry | 0.5-5 |

Procedure: Entacapone, mannitol and beta cyclodextrin were co-sifted and mixed with microcrystalline cellulose, croscarmellose sodium and colloidal silicon dioxide in double cone blender. Magnesium stearate was mixed with above pre-mix in a double cone blender. Half of this mixture was compacted through a roll compactor and sizing was carried out to break flakes in to granules using a multi mill. The remaining half of the mixture was also compacted through a roll compactor along with fines of first half and sizing was done using a multimill to obtain granules of desired size. The granules were mixed with hydrogenated vegetable oil, sodium starch glycollate, colloidal silicon dioxide and talc. The granules were lubricated with magnesium stearate and the final blend was compressed in to tablets using suitable tooling and coated with aqueous dispersion of Opadry.

While the invention has been described in terms of its specific embodiments, certain modifications and equivalents will be apparent to those skilled in the art and are intended to be included within the scope of the invention.

We claim:

1. A method of making a pharmaceutical composition, the method comprising providing particles of entacapone or salts thereof having a particle size (D₉₀) of 40 microns or less; forming a mixture by mixing the particles of entacapone or salts thereof with one or more pharmaceutically acceptable excipients; and forming the mixture into a pharmaceutical dosage form.

2. The method of claim 1, further comprising reducing the particle size of the particles of entacapone or salts thereof in a particle size reducing operation.

3. The method of claim 2, wherein the particle size reducing operation comprises one or both of chemical and mechanical methods.

4. The method of claim 3, wherein the chemical method comprises one or more of a solvent crystallization, chemical synthesis, modified crystal engineering, freeze-drying, and spray drying.

5. The method of claim 3, wherein the mechanical method comprises one or more of ball mill, nano mill, attritor mill, vibratory mill, sand mill, bead mill, jet mill, ultrasonication, and microfluidization.

6. The method of claim 2, wherein the particle size reducing operation reduces the size of the particles of entacapone or salts thereof to have a particle size (D₉₀) that is 40 microns or less.

7. The method of claim 1, wherein the pharmaceutically acceptable excipients comprises one or more of surfactants, binders, fillers, lubricants, disintegrants, and glidants.

8. The method of claim 1, wherein the pharmaceutical dosage form is a monolayered tablet, tablet in a tablet, a bilayered tablet, a caplet, a minitablet, a capsule, a tablet in a capsule, granules in a capsule, pellets, pellets in a capsule, beads, discs, pills, spheroids, sachet, minitablets in capsules, powder or a suspension.

9. A pharmaceutical composition comprising particles of entacapone or salts thereof, wherein the particles have a particle size (D₉₀) of 40 microns or less.

10. The pharmaceutical composition of claim 9, wherein the composition further comprises one or more pharmaceutically acceptable excipients comprising surfactants, binders, fillers, lubricants, disintegrants, and glidants.

11. The pharmaceutical composition of claim 9, wherein the composition is in the form of a monolayered tablet, tablet in a tablet, a bilayered tablet, a caplet, a minitablet, a capsule, a tablet in a capsule, granules in a capsule, pellets, pellets in a capsule, beads, discs, pills, spheroids, sachet, minitablets in capsules, powder or a suspension.

12. A pharmaceutical composition comprising granules of entacapone, wherein the granules have a size of 900 microns or less.

13. The pharmaceutical composition of claim 12, wherein the composition further comprises one or more pharmaceutically acceptable excipients comprising surfactants, binders, fillers, lubricants, disintegrants, and glidants.

14. The pharmaceutical composition of claim 12, wherein the composition is in the form of a monolayered tablet, tablet in a tablet, a bilayered tablet, a caplet, a minitablet, a capsule, a tablet in a capsule, granules in a capsule, pellets, pellets in a capsule, beads, discs, pills, spheroids, sachet, minitablets in capsules, powder or a suspension.

15. A pharmaceutical composition comprising particles of entacapone or salts thereof having a particle size (D₉₀) of 40 microns or less, wherein the composition has at least 80% dissolution of entacapone or salts thereof within 30 minutes when tested in Apparatus 2 (USP, Dissolution, paddle, 50 rpm) using 900 ml of pH 5.5 phosphate buffer at 37° C.±0.5° C.

16. The pharmaceutical composition of claim 15, wherein the composition further comprises one or more pharmaceutically acceptable excipients comprising surfactants, binders, fillers, lubricants, disintegrants, and glidants.

17. A pharmaceutical composition comprising entacapone or salts thereof and a wetting agent.

18. The pharmaceutical composition of claim 17, wherein the wetting agent is a surface-active agent.

19. The pharmaceutical composition of claim 18, wherein the surface-active agent is anionic, cationic, or non-ionic.

20. The pharmaceutical composition of claim 19, wherein the anionic surface active agent comprises one or more of sodium dodecyl sulfate, sodium laurate, dialkylsodium sulfosuccinates, sodium stearate, potassium stearate, and sodium oleate.

21. The pharmaceutical composition of claim 19, wherein the cationic surface active agent comprises one or more of benzalkonium chloride, bis-2-hydroxyethyl oleyl amine, benzethonium chloride, and cetrimide.

22. The pharmaceutical composition of claim 19, wherein the non-ionic surface-active agent comprises one or more of polyoxyethylene sorbitan fatty acid esters, fatty alcohols, and glyceryl esters.

23. The pharmaceutical composition of claim 17, wherein the composition further comprises one or more pharmaceutically acceptable excipients comprising binders, fillers, lubricants, disintegrants, and glidants.

24. The pharmaceutical composition of claim 17, wherein the composition is in the form of a monolayered tablet, tablet in a tablet, a bilayered tablet, a caplet, a minitablet, a capsule, a tablet in a capsule, granules in a capsule, pellets, pellets in a capsule, beads, discs, pills, spheroids, sachet, minitablets in capsules, powder or a suspension.

25. A pharmaceutical composition comprising entacapone or salts thereof and one or more cyclodextrins or derivatives thereof.

26. The pharmaceutical composition of claim 25, wherein the entacapone is present in admixture with cyclodextrins or derivatives thereof.

27. The pharmaceutical composition of claim 25, wherein the entacapone is present in the form of complex with cyclodextrins or derivatives thereof.

28. The pharmaceutical composition of claim 25, wherein the cyclodextrins comprises one or more of β-cyclodextrin, α-cyclodextrin, γ-cyclodextrins, hydroxypropyl-α-cyclodextrin, hydroxypropyl-β-cyclodextrin, dimethyl-β-cylcodextrin, 2-hydroxyethyl β-cyclodextrin, trimethyl-β-cyclodextrin, and sulfonated cyclodextrins.

29. The pharmaceutical composition of claim 25, wherein the composition further comprises one or more pharmaceutically acceptable excipients comprising surfactants, binders, fillers, lubricants, disintegrants, and glidants.

30. The pharmaceutical composition of claim 25, wherein the composition is in the form of a monolayered tablet, tablet in a tablet, a bilayered tablet, a caplet, a minitablet, a capsule, a tablet in a capsule, granules in a capsule, pellets, pellets in a capsule, beads, discs, pills, spheroids, sachet, minitablets in capsules, powder or a suspension.
